(19) United States
(12) Patent Application Publication
(10) Pub. No.: US 2024/0358006 A1
(43) Pub. Date: Oct. 31, 2024

(54) TRANSGENIC TEAL TETRA

(71) Applicant: SPECTRUM BRANDS, INC., MIDDLETON, WI (US)

(72) Inventors: Aidas NASEVICIUS, Tampa, FL (US); John Edward FOX, Middleton, WI (US)

(21) Appl. No.: 18/688,495

(22) PCT Filed: Aug. 26, 2022
(86) PCT No.: PCT/US2022/041639

§ 371 Date: Mar. 1, 2024

Related U.S. Application Data
(60) Provisional application No. 63/240,419, filed on Sep. 3, 2021.

(51) Int. Cl. A01K 67/0275; C12N 15/90

(52) U.S. Cl. CPC A01K 67/0275; C12N 15/907; A01K 2217/052; A01K 2227/40; A01K 2267/01

(57) ABSTRACT

The present invention relates to transgenic ornamental fish, as well as methods of making such fish by in vitro fertilization. Also disclosed are methods of establishing a population of such transgenic fish and methods of providing them to the ornamental fish industry for the purpose of marketing.

### CROSS-REFERENCE TO RELATED APPLICATIONS

This application is being filed on Aug. 26, 2022, as a PCT International application and claims priority to and the benefit of U.S. Provisional Patent Application No. 63/240,419 filed Sep. 3, 2021, the disclosure of which is incorporated by reference herein in its entirety. To the extent appropriate a claim for priority is made to the above referenced application.

### FIELD OF THE INVENTION

This invention relates to transgenic fish, particularly teal transgenic fish.

### INTRODUCTION

Transgenic technology involves the transfer of a foreign gene into a host organism enabling the host to acquire a new and inheritable trait. Transgenic technology has many potential applications. For example, it can be used to introduce a transgene into a fish in order to create new varieties of fish. There are many ways of introducing a foreign gene into fish, including: microinjection, electroporation, sperm-mediated gene transfer, gene bombardment or gene gun, liposome-mediated gene transfer, and the direct injection of DNA into fish tissue.

Early transgenic fish used a chimeric gene construct consisting of a mouse metallothionein gene promoter and a human growth hormone gene. Most of the early transgenic fish studies have concentrated on growth hormone gene transfer with an aim of generating fast growing fish. While a majority of early attempts used heterologous growth hormone genes and promoters and failed to produce these fish, enhanced growth of transgenic fish has been demonstrated in several fish species including Atlantic salmon, several species of Pacific salmons, and loach.

The black skirt tetra (Gymnocorymbus ternetzi) has been commercially cultured in the United States at least as early as 1950. However, for the ornamental fish industry the dark striped pigmentation of the adult black skirt tetra does not aid in the efficient display of the various colors. The albino black skirt tetra, or “white skirt tetra” is a variant that arose during domestication and shows decreased pigmentation. The availability of such fish having modified pigmentation for transgenesis with fluorescent proteins would result in better products for the ornamental fish industry due to better visualization of fluorescent colors.

Many fluorescent proteins are known in the art and have been used to investigate various cellular processes, including fluorescent proteins exhibiting various green, red, yellow, blue, or purple colors. Although transgenic experiments involving fluorescent proteins have provided new markers and reporters for transgenesis, progress in the field of developing and producing ornamental fish that express such proteins has been limited.

Moreover, the ability to provide transgenic fish that display colors beyond the classic green, red, yellow, blue or purple colors are difficult to predict based on several factors, including but not limited to: the species, combination of colors being used, gestation cycle, and consistency of color through multiple generations (such as F1, F2, F3, etc.). Thus, there is a need for a color crossed transgenic fish which will retain the color visualization through multiple generations that retains a consistent hybrid color.

### Transgenic Teal Tetra

In certain embodiments, the present disclosure concerns making transgenic fluorescent fish and providing such fish to the ornamental fish industry.

In some embodiments, transgenic fish or methods of making transgenic fish are provided. In certain aspects, the transgenic fish are fertile, transgenic, fluorescent fish. In at least one example embodiment, the fish for use with the disclosed constructs and methods is the white skirt tetra. Tetra skin color is determined by pigment cells in their skin, which contain pigment granules called melanosomes (black or brown color), xanthosomes (yellow color), erythrosomes (orange or red color), or iridosomes (iridescent colors, including white color). The number, size, and density of the pigment granules per pigment cell influence the color of the fish skin. White skirt tetra have diminished number, size, and density of melanosomes and hence have lighter skin when compared to the wild type black skirt tetra.

In some example embodiments there are provided transgenic tetra or progeny thereof comprising specific transgenic integration events, referred to herein as transformation events. These fish are of particular interest because, for example, they embody an aesthetically pleasing teal color. Transgenic fish comprising these specific transgenic events may be homozygous or heterozygous (including, for example, hemizygous) for each of the transformation event(s). Homozygous fish bred with fish lacking a transformation event will in nearly all cases produce 100% heterozygous offspring. Eggs, sperm, and embryos comprising these specific transgenic events are also included as part of the invention.

In at least one example embodiment, Teal tetras comprise 2 expression cassettes. Both cassettes can be homozygous. In other embodiments, each of the cassettes can be heterozygous. In alternative embodiments one expression cassette can be homozygous and the other expression cassette is heterozygous. It should be appreciated that fish of different zygosities will produce different ratios of offspring when bred.

In at least one example embodiment, a tetra will comprise a specific transgenic integration event, a teal transgenic tetra or progeny thereof is provided comprising chromosomally integrated transgenes, wherein the tetra comprises the “Teal tetra 1 transformation event,” sperm comprising the Teal tetra 1 transformation event having been deposited as ATCC accession no. PTA-127007. The chromosomally integrated transgenes comprise a chromosomally integrated expression cassette encoding a green fluorescent protein, a chromosomally integrated expression cassette encoding blue fluorescent protein, and/or a chromosomally integrated expression cassette encoding blue chromoprotein or blue-colored fluorescent protein

In some example embodiments, the teal tetra will comprise a green fluorescent protein, or a portion thereof and/or a blue fluorescent protein or a portion thereof with a blue non-fluorescent chromoprotein. In at least these example embodiments, a transgenic tetra is a fertile, transgenic tetra. In more specific aspects, such a tetra is a transgenic White skirt tetra. Such a transgenic tetra may be homozygous or heterozygous (including, for example, hemizygous) for the transgenes or integrated expression cassette(s).

The chromosomally integrated transgenes may be present on one integrated expression cassette or two or more integrated expression cassettes. In some example embodiments, the teal tetra will comprise a green fluorescent protein, or a portion thereof and a blue fluorescent protein, or a portion thereof and a blue non-fluorescent chromoprotein. In at least these example embodiments, a transgenic blue tetra is a fertile, transgenic tetra and is crossed with a transgenic green tetra which is a fertile, transgenic tetra. In more specific aspects, the progeny of such a cross is a transgenic Teal tetra. In at least these example embodiments, such a transgenic tetra may be homozygous or heterozygous (including, for example, hemizygous) for the transgenes or integrated expression cassette(s).

Also disclosed are methods of providing a transgenic tetra comprising the Teal tetra 1 transformation event to the ornamental fish market. In some embodiments, the method comprises obtaining a transgenic tetra or progeny thereof comprising chromosomally integrated transgenes, wherein the tetra comprises the “Teal tetra 1 transformation event,” sperm comprising the Teal tetra 1 transformation event having been deposited as ATCC accession no. PTA-127007, and distributing the fish to the ornamental fish market. Such fish may be distributed by a grower to a commercial distributor, or such fish may be distributed by a grower or a commercial distributor to a retailer such as, for example, a multi-product retailer having an ornamental fish department.

In some aspects, methods of producing a transgenic tetra are provided comprising: (a) obtaining a tetra that exhibits fluorescence and comprises one or more chromosomally integrated transgenes or expression cassettes, wherein the tetra comprises the “Teal tetra 1 transformation event,” sperm comprising the Teal tetra 1 transformation event having been deposited as ATCC accession no. PTA-127007; and (b) breeding the obtained tetra with a second tetra to provide a transgenic tetra comprising the Teal tetra 1 transformation event. The second tetra may be a transgenic or non-transgenic tetra. In further embodiments, also provided are methods of producing a transgenic organism, the method comprising using sperm comprising the Teal tetra 1 transformation, such sperm having been deposited as ATCC accession no. PTA-127007, to produce transgenic offspring. Such offspring may be, for example, a tetra, a species of the Gymnocorymbus genus, a fish species or genus related to tetra, or another fish species or genus. In some aspects, the fish may be produced using in vitro fertilization techniques known in the art or described herein.

As used in this specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Any embodiment of any of the present methods, kits, and compositions may consist of or consist essentially of—rather than comprise/include/contain/have—the described features and/or steps. Thus, in any of the claims, the term “consisting of” or “consisting essentially of” may be substituted for any of the open-ended linking verbs recited above, in order to change the scope of a given claim from what it would otherwise be using the open-ended linking verb.

Other objects, features and advantages of the present disclosure will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### DETAILED DESCRIPTION

### Transgenic Fish

In some aspects, the invention regards transgenic fish. Methods of making transgenic fish are described in, for example, U.S. Pat. Nos. 7,135,613; 7,700,825; 7,834,239, each of which is incorporated by reference in its entirety. For example, a transgenic Tetra may be generated using an expression cassette encoding green fluorescent protein (GFP), such as ZsGreen1. In another example, a transgenic orange Tetra may be generated using an expression cassette encoding orange fluorescent protein (YFP), such as ZsYellow1. In another example, a transgenic red Tetra may be generated using an expression cassette encoding red fluorescent protein (RFP), such as DsRed2. In another example, a transgenic purple Tetra may be generated using an expression cassette encoding purple fluorescent protein (PFP), such as FP635. In another example, a transgenic blue Tetra may be generated using an expression cassette encoding blue fluorescent protein (BFP), such as TagBFP, or TagBFP in combination with aeCP597.

In related embodiments blue colored proteins are selected from a group comprising aeCP597, eqFP670, E2-Crimson, mPlum, mRaspberry, mCardinal, mNeptune and combinations thereof.

In other related embodiments the BFP is selected from a group comprising SBFP2, TagBFP, TagBFP2, shBFP, Azurite, eBFP2, oxBFP, mBlueberry2, mKalama1, eBFP 1.2, eBFP.A5, moxBFP, mTagBFP, mTagBFP2 or combinations thereof. In related embodiments these BFPs are combined with at least one blue colored protein selected from a group comprising aeCP597, eqFP670, E2-Crimson, mPlum, mRaspberry, mCardinal, mNeptune. mNeptune2, Neptune, TagRFP657, TagRFP658, Maroon0.1, mMaroon1, mGrape2, mGrape3, mCarmine, mKelly, mKelly2 and combinations thereof.

In other related embodiments the blue chromoprotein is selected from a group comprising CP-580 from Acropora hyacinthus, CP-580 from Acropora aculeus, CP-588 from Acropora formosa (now called A. muricata), CP-588 from Rhizostoma pulmo and Cassiopeia xamachana, CP-588 Acropora millepora, CP-590 from Montipora efflorescens, CP-592 from Montipora species, CP-595 from Anemonia sulcate, CP-597 from Anemonia equina, CP-597 from Anemonia majano, aeBlue, Ultramarine, meffBlue (Rtms5) and combinations thereof.

It should be appreciated that the chromoprotein may exist to enhance the fluorescent characteristics of the Teal tetra without the ability to fluoresce when not combined with another fluorescent protein. Further, blue chromoproteins and few blue looking fluorescent proteins will exhibit blue fluorescent characteristics in white light. Although not wanting to be bound by any particular theory, a green fluorescent protein mixed with blue-looking protein (chromoprotein, or blue-looking red/far-red fluorescent protein) will yield the physical teal visualization. Some exemplary embodiments, the blue fluorescent protein does not contribute to the teal coloration observed in white light, but rather provides the blue fluorescence in UV light.

It is preferred that fish belonging to species and varieties of fish of commercial value, particularly commercial value within the ornamental fish industry, be used. Such fish include but are not limited to catfish, zebrafish and other danios, medaka, carp, tilapia, goldfish, tetras, barbs, sharks (family Cyprinidae), angelfish, loach, koi, glassfish, catfish, discus, eel, tetra, goby, gourami, guppy, Xiphophorus, hatchet fish, Molly fish, or pangasius. A particular fish for use in the context of the invention is a tetra, Gymnocorymbus ternetzi. Tetra are increasingly popular ornamental animals and would be of added commercial value in various colors. Tetra embryos are easily accessible and nearly transparent. A fish that is of particular use with the disclosed constructs and methods is the White Skirt Tetra.

Fertilization from Frozen Sperm

Fish sperm freezing methods are well-known in the art; see, e.g., Walker and Streisinger (1983) and Draper and Moens (2007), both of which are incorporated herein by reference in their entireties. To obtain the transgenic fish disclosed herein, frozen tetra sperm may be used to fertilize eggs.

Briefly, one or two breeding pairs of tetra should be placed in a shoebox (or equivalent container) with an artificial spawning mat. The water level in the shoebox should be ^{˜}2-3 inches and kept at 75-85° F. Low salinity (conductivity 100-200 uS/cm) and slight acidity (^{˜}pH 6.9) promote spawning. The fish may be exposed to a natural or artificial light cycle; the photoperiod starts at 8 am and ends at 10 pm. The following morning, remove and discard the eggs. Tetra may be anesthetized by immersion in tricaine solution at 16 mg/100 mL water. After gill movement has slowed, remove one female, rinse it in water, and gently blot the belly damp-dry with a paper towel. The eggs should not be exposed to water as this will prevent fertilization. Gently squeeze out the eggs onto a slightly concave surface by applying light pressure to the sides of the abdomen with, for example, a thumb and index finger and sliding the fingers to the genital pore. Ready to spawn females will release the eggs extremely easily, and care should be taken not to squeeze the eggs out while blotting the fish. Good eggs are yellowish and translucent; eggs that have remained in the female too long appear white and opaque. The females will release the eggs only for an hour or so. Eggs from several females may be pooled; the eggs can be kept unfertilized for several minutes. The sperm is thawed at 33° C. in a water bath for 18-20 seconds. Additionally, 70 μl room temperature Hanks solution is added to the vial and mixed. The sperm is then immediately added to the eggs and gently mixed. The sperm and eggs are activated by adding 750 μl of fish water and mixing.

The mixture is incubated for 5 minutes at room temperature. The dish is then filled with fish water and incubated at 28° C. After 2-3 hours, fertile embryos are transferred to small dishes where they are further cultured.

Parichy and Johnson, 2001, which is incorporated by reference in its entirety, provides additional examples regarding in vitro fertilization.

The invention further encompasses progeny of a transgenic fish containing the Teal tetra 1 transformation event, as well as such transgenic fish derived from a transgenic fish egg, sperm cell, embryo, or other cell containing a genomically integrated transgenic construct. “Progeny,” as the term is used herein, can result from breeding two transgenic fish of the invention, or from breeding a first transgenic fish of the invention to a second fish that is not a transgenic fish of the invention. In the latter case, the second fish can, for example, be a wild-type fish, a specialized strain of fish, a mutant fish, or another transgenic fish. The hybrid progeny of these matings have the benefits of the transgene for fluorescence combined with the benefits derived from these other lineages.

The simplest way to identify fish containing the Teal tetra 1 transformation event is by visual inspection, as the fish in question would be a teal color and have fluorescent characteristics, which are immediately distinguishable from non-transgenic fish.

### EXAMPLES

Certain embodiments of the invention are further described with reference to the following examples. These examples are intended to be merely illustrative of the invention and are not intended to limit or restrict the scope of the present disclosure in any way and should not be construed as providing conditions, parameters, reagents, or starting materials that must be utilized exclusively in order to practice the art of the present disclosure.

Transgenic fish exhibiting a teal color are provided. The specific transgenic events embodied in these fish are designated Teal tetra 1. Sperm from these fish can be obtained, for example, from the progeny of crossing a blue fluorescent tetra and a green fluorescent tetra. It should be appreciated that the ability to provide transgenic fish that display colors that comprise “color-crossed” combinations would not be readily apparent to one skilled in the art, nor is the ability to obtain a novel color readily obtained without undue experimentation. This is particularly evidence when desiring multiple generations of having consistent visualization of the Teal Tetra transformation event. The Examples below show various related examples of Teal tetras that can be obtained.

In some example embodiments, an expression cassette encoding green fluorescent protein (GFP) and an expression cassette encoding for the blue fluorescent protein (BFP), such as TagBFP, or TagBFP in combination with aeCP597 are injected, thereby encoding for both green and blue colors, but the Tetra comprises a Teal visualization effect. It should be appreciated that TagBFP, TagBFP2, shBFP, Azurite, eBFP2, oxBFP, mBlueberry2, mKalama1 or combinations thereof could be used alone or in combination with at least one blue colored protein selected from a group comprising aeCP597, eqFP670, E2-Crimson, mPlum, mRaspberry, mCardinal, mNeptune and combinations thereof.

In some example embodiments, an expression cassette encoding green fluorescent protein (GFP) and an expression cassette encoding for the blue fluorescent protein (BFP), such as TagBFP, or TagBFP in combination with an alternative blue-colored protein are injected, thereby encoding for both colors, but the Tetra comprising a Teal visualization effect.

In other embodiments, a specific transgenic integration event, a GFP and BFP Tetra or progeny thereof is provided comprising chromosomally integrated transgenes that may be present on one integrated expression cassette for each fluorescent protein or two or more integrated expression cassettes for each fluorescent protein. In certain aspects, such a transgenic Tetra is a fertile, transgenic Tetra. Such a transgenic Tetra may be homozygous or heterozygous (including, for example, hemizygous) for the transgenes or integrated expression cassette(s).

Also disclosed are methods of providing a transgenic Tetra comprising the Teal Tetra 1 transformation event to the ornamental fish market. In some embodiments, the method comprises obtaining a transgenic Tetra or progeny thereof comprising chromosomally integrated transgenes, wherein the Tetra comprises the “Teal Tetra 1 transformation event,” cryopreserved sperm being deposited at the ATCC under accession no. PTA-127007, and distributing the fish to the ornamental fish market. Such fish may be distributed by a grower to a commercial distributor, or such fish may be distributed by a grower or a commercial distributor to a retailer such as, for example, a multi-product retailer having an ornamental fish department.

### Example 1—Teal Transgenic Tetra

Transgenic fish exhibiting a teal color are provided. The specific transgenic events embodied in these fish are designated Teal tetra 1. Sperm from these fish may be used to fertilize tetra eggs and thereby breed transgenic tetra that comprise these specific transgenic integration events. Sperm from this line is being deposited at the American Type Culture Collection, 10801 University Blvd, Patent Depository, Manassas, VA 20110-2209, under the provisions of the Budapest Treaty as “Teal tetra 1” (accession no. PTA-127007).

The fluorescent transgenic fish have use as ornamental fish in the market. Stably expressing transgenic lines can be developed, in at least one example embodiment, by breeding a transgenic individual with a wild-type fish, mutant fish, or another transgenic fish. The desired transgenic fish can be distinguished from non-transgenic fish by observing the fish in white light, sunlight, ultraviolet light, blue light, or any other useful lighting condition that allows visualization of the teal color of the transgenic fish.

### Example 2 Teal Transgenic Tetra

Transgenic fish exhibiting a teal color are provided. The specific transgenic events embodied in these fish are designated Teal tetra 1. Sperm from these fish may be used to fertilize tetra eggs and thereby breed transgenic tetra that comprise these specific transgenic integration events. Sperm from this line is being deposited at the American Type Culture Collection, 10801 University Blvd, Patent Depository, Manassas, VA 20110-2209, under the provisions of the Budapest Treaty as “Teal tetra 1” (accession no. PTA-127007).

Stably expressing transgenic lines can be developed by breeding a transgenic individual with a wild-type fish, mutant fish, or another transgenic fish. The desired transgenic fish can be distinguished from non-transgenic fish by observing the fish in white light, sunlight, ultraviolet light, blue light, or any other useful lighting condition that allows visualization of the teal color of the transgenic fish. In at least this example embodiment a teal transgenic fish comprising a teal visualization would be hemizygous and comprise a blue fluorescent protein and a green fluorescent protein the sperm of the progeny of the transgenic fish, the percentage of identical genetic similarity being at least 95% of the sperm having the Teal tetra 1 transformation event (ATCC accession no. PTA-127007). In at least this example embodiment a transgenic fish having the Teal tetra 1 transformation event is hemizygous for both green and blue colors. In at least these example embodiments, the expected progeny when fertilizing wild type eggs would be 25% of each wild type, green, blue, and teal.

In related embodiments the Teal Tetra 1 is formed from breeding a tetra comprising a chromosomally integrated expression cassette encoding a green fluorescent protein with a tetra comprising a chromosomally integrated expression cassette encoding blue fluorescent protein and a chromosomally integrated expression cassette encoding blue chromoprotein or blue-colored fluorescent protein. The progeny of such breeding results in a fertile next generation fish line. In some instances a sterile progeny line is desired and allows for the Teal Tetra 1 from being sourced from, for example, a specific generation line such as (F1, F2, F3, etc.).

In another example embodiment, a Teal Tetra 1 is formed from breeding a tetra comprising a chromosomally integrated expression cassette encoding a green fluorescent protein, a chromosomally integrated expression cassette encoding blue fluorescent protein, and/or a chromosomally integrated expression cassette encoding blue chromoprotein or blue-colored fluorescent protein with a second tetra comprising a chromosomally integrated expression cassette encoding a green fluorescent protein, a chromosomally integrated expression cassette encoding blue fluorescent protein, and/or a chromosomally integrated expression cassette encoding blue chromoprotein or blue-colored fluorescent protein. The progeny of such breeding results in a fertile next generation fish line. In some example embodiments the present disclosure provides a sterile progeny and allows for the Teal Tetra 1 from being sourced from, for example, a specific generation line such as (F1, F2, F3, etc.).

In another example embodiment, a Teal Tetra 1 is formed from breeding a tetra comprising at least one of a chromosomally integrated expression cassette encoding a green fluorescent protein, a chromosomally integrated expression cassette encoding blue fluorescent protein, and/or a chromosomally integrated expression cassette encoding blue chromoprotein or blue-colored fluorescent protein with a second tetra comprising a chromosomally integrated expression cassette encoding a green fluorescent protein. The progeny of such breeding results in a fertile next generation fish line. In some example embodiments the present disclosure provides a sterile progeny and allows for the Teal Tetra 1 from being sourced from, for example, a specific generation line such as (F1, F2, F3, etc.).

In another example embodiment, a Teal Tetra 1 is formed from breeding a tetra comprising a chromosomally integrated expression cassette encoding a green fluorescent protein, a chromosomally integrated expression cassette encoding blue fluorescent protein, and/or a chromosomally integrated expression cassette encoding blue chromoprotein or blue-colored fluorescent protein with a second tetra comprising a chromosomally integrated expression cassette encoding blue fluorescent protein, and/or a chromosomally integrated expression cassette encoding blue chromoprotein or blue-colored fluorescent protein. The progeny of such breeding results in a fertile next generation fish line. In some example embodiments the present disclosure provides a sterile progeny and allows for the Teal Tetra 1 from being sourced from, for example, a specific generation line such as (F1, F2, F3, etc.).

In another example embodiment, a Teal Tetra 1 is formed from breeding a tetra comprising a chromosomally integrated expression cassette encoding a green fluorescent protein, a chromosomally integrated expression cassette encoding blue fluorescent protein, and/or a chromosomally integrated expression cassette encoding blue chromoprotein or blue-colored fluorescent protein with a second tetra being a wild type (non-transgenic) white skirt tetra. In at least this example embodiment, the progeny will 25% Teal. The progeny of such breeding results in a fertile next generation fish line. In some example embodiments the present disclosure provides a sterile progeny and allows for the Teal Tetra 1 from being sourced from, for example, a specific generation line such as (F1, F2, F3, etc.).

The fluorescent transgenic fish described above are also valuable in the market for scientific research tools because they can be used for embryonic studies such as tracing cell lineage and cell migration. Additionally, these fish can be used to mark cells in genetic mosaic experiments and in fish cancer models.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the methods described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope, and concept of the invention as defined by the appended claims.

The following numbered clauses define further example aspects and features of the present disclosure:

1. A transgenic tetra comprising a chromosomally integrated expression cassette encoding a green fluorescent protein, a chromosomally integrated expression cassette encoding blue fluorescent protein, and/or a chromosomally integrated expression cassette encoding blue chromoprotein or blue-colored fluorescent protein, wherein the tetra comprises the “Teal tetra 1 transformation event,” sperm comprising the Teal tetra 1 transformation event having been deposited as ATCC accession no. PTA-127007.

2. The transgenic tetra of clause 1, further defined as a fertile, transgenic tetra.

3. The transgenic tetra of clause 1, further defined as a sterile transgenic Teal Tetra.

4. The transgenic tetra of clause 1, wherein the fish is homozygous for at least one of the integrated expression cassettes.

5. The transgenic tetra of clause 1 or clause 4, wherein the fish is heterozygous for at least one of the integrated expression cassettes.

6. A method of providing a transgenic tetra to the ornamental fish market, comprising obtaining a transgenic tetra in accordance with clause 1, and distributing the fish to the ornamental fish market.

7. The method of clause 6, wherein the fish are distributed by a grower to a commercial distributor.

8. The method of clause 6, wherein the fish are distributed by a grower or a commercial distributor to a retailer.

9. The method of clause 8, wherein the retailer is a multi-product retailer having an ornamental fish department.

10. A method of producing a transgenic tetra comprising:
(a) obtaining a tetra that comprises a chromosomally integrated expression cassette encoding a green fluorescent protein, a chromosomally integrated expression cassette encoding blue fluorescent protein, and/or a chromosomally integrated expression cassette encoding blue chromoprotein or blue-colored fluorescent protein, wherein the tetra comprises the “Teal tetra 1 transformation event,” sperm comprising the Teal tetra 1 transformation event having been deposited as ATCC accession no. PTA-127007; and
(b) breeding the obtained tetra with a second tetra to provide a transgenic tetra comprising the Teal tetra 1 transformation event.

11. The method of clause 10, wherein the second tetra is a non-transgenic tetra.

12. A progeny of a transgenic tetra of clause 1 that comprises a chromosomally integrated expression cassette encoding a green fluorescent protein, a chromosomally integrated expression cassette encoding blue fluorescent protein, and/or a chromosomally integrated expression cassette encoding blue chromoprotein or blue-colored fluorescent protein wherein the tetra and progeny comprises the “Teal tetra 1 transformation event,” sperm comprising the Teal tetra 1 transformation event having been deposited as ATCC accession no. PTA-127007.

13. The progeny fish of clause 12, further defined as a fertile, transgenic tetra.

14. The progeny fish of clause 12, further defined as a sterile Teal Tetra.

15. The progeny fish of clause 12, wherein the fish is homozygous for at least one of the GFP or BFP integrated expression cassettes.

16. The progeny fish of clause 12, wherein the fish is heterozygous for the integrated expression cassettes.

17. A method of providing a transgenic fish to the ornamental fish market, comprising obtaining a progeny fish in accordance with clause 12, and distributing the fish to the ornamental fish market.

18. The method of clause 17, wherein the fish are distributed by a grower to a commercial distributor.

19. The method of clause 17, wherein the fish are distributed by a grower or a commercial distributor to a retailer.

20. The method of clause 19, wherein the retailer is a multi-product retailer having an ornamental fish department.

21. A method of producing a transgenic fish comprising:
(a) obtaining a transgenic fish in accordance with clause 12; and
(b) breeding the obtained fish with a second fish to provide a transgenic fish comprising the Teal tetra 1 transformation event transformation event.

22. The method of clause 21, wherein the second fish is a non-transgenic fish.

23. A transgenic tetra comprising a chromosomally integrated expression cassette encoding a green fluorescent protein, a chromosomally integrated expression cassette encoding blue fluorescent protein, and/or a chromosomally integrated expression cassette encoding blue chromoprotein or blue-colored fluorescent protein, wherein the tetra comprises the “Teal tetra 1 transformation event”.

24. The transgenic tetra of clause 23, further defined as a fertile, transgenic tetra.

25. The transgenic tetra of clause 23, further defined as a sterile transgenic Teal Tetra.

26. The transgenic tetra of clause 23, wherein the fish is homozygous for at least one of the integrated expression cassettes.

27. The transgenic tetra of clause 23 or clause 26, wherein the fish is heterozygous for at least one of the integrated expression cassettes.

28. The transgenic tetra of any one of clauses 23-27, wherein the Teal tetra 1 transformation event having been deposited as ATCC accession no. PTA-127007.

The above specification, examples and data provide a complete description of the manufacture and use of the composition of the invention. Since many embodiments of the invention can be made without departing from the spirit and scope of the invention, the invention resides in the claims hereinafter appended.

1. A transgenic tetra comprising a chromosomally integrated expression cassette encoding a green fluorescent protein, a chromosomally integrated expression cassette encoding blue fluorescent protein, and/or a chromosomally integrated expression cassette encoding blue chromoprotein or blue-colored fluorescent protein, wherein the tetra comprises the “Teal tetra 1 transformation event,” sperm comprising the Teal tetra 1 transformation event having been deposited as ATCC accession no. PTA-127007.

2. The transgenic tetra of claim 1, further defined as a fertile, transgenic tetra.

3. The transgenic tetra of claim 1, further defined as a sterile transgenic Teal Tetra.

4. The transgenic tetra of claim 1, wherein the fish is homozygous for at least one of the integrated expression cassettes.

5. The transgenic tetra of claim 1, wherein the fish is heterozygous for at least one of the integrated expression cassettes.

6. A method of providing a transgenic tetra to the ornamental fish market, comprising obtaining a transgenic tetra in accordance with claim 1, and distributing the fish to the ornamental fish market.

7. The method of claim 6, wherein the fish are distributed by a grower to a commercial distributor.

8. The method of claim 6, wherein the fish are distributed by a grower or a commercial distributor to a retailer.

9. The method of claim 8, wherein the retailer is a multi-product retailer having an ornamental fish department.

10. A method of producing a transgenic tetra comprising:
(a) obtaining a tetra that comprises a chromosomally integrated expression cassette encoding a green fluorescent protein, a chromosomally integrated expression cassette encoding blue fluorescent protein, and/or a chromosomally integrated expression cassette encoding blue chromoprotein or blue-colored fluorescent protein, wherein the tetra comprises the “Teal tetra 1 transformation event,” sperm comprising the Teal tetra 1 transformation event having been deposited as ATCC accession no. PTA-127007; and
(b) breeding the obtained tetra with a second tetra to provide a transgenic tetra comprising the Teal tetra 1 transformation event.

11. The method of claim 10, wherein the second tetra is a non-transgenic tetra.

12. A progeny of a transgenic tetra of claim 1 that comprises a chromosomally integrated expression cassette encoding a green fluorescent protein, a chromosomally integrated expression cassette encoding blue fluorescent protein, and/or a chromosomally integrated expression cassette encoding blue chromoprotein or blue-colored fluorescent protein wherein the tetra and progeny comprises the “Teal tetra 1 transformation event,” sperm comprising the Teal tetra 1 transformation event having been deposited as ATCC accession no. PTA-127007.

13. The progeny fish of claim 12, further defined as a fertile, transgenic tetra.

14. The progeny fish of claim 12, further defined as a sterile Teal Tetra.

15. The progeny fish of claim 12, wherein the fish is homozygous for at least one of the GFP or BFP integrated expression cassettes.

16. The progeny fish of claim 12, wherein the fish is heterozygous for the integrated expression cassettes.

17. A method of providing a transgenic fish to the ornamental fish market, comprising obtaining a progeny fish in accordance with claim 12, and distributing the fish to the ornamental fish market.

18. The method of claim 17, wherein the fish are distributed by a grower to a commercial distributor.

19. The method of claim 17, wherein the fish are distributed by a grower or a commercial distributor to a retailer.

20. The method of claim 19, wherein the retailer is a multi-product retailer having an ornamental fish department.

21. A method of producing a transgenic fish comprising:
(a) obtaining a transgenic fish in accordance with claim 12; and
(b) breeding the obtained fish with a second fish to provide a transgenic fish comprising the Teal tetra 1 transformation event transformation event.

22. The method of claim 21, wherein the second fish is a non-transgenic fish.

23. A transgenic tetra comprising a chromosomally integrated expression cassette encoding a green fluorescent protein, a chromosomally integrated expression cassette encoding blue fluorescent protein, and/or a chromosomally integrated expression cassette encoding blue chromoprotein or blue-colored fluorescent protein, wherein the tetra comprises the “Teal tetra 1 transformation event”.

24. The transgenic tetra of claim 23, further defined as a fertile, transgenic tetra.

25. The transgenic tetra of claim 23, further defined as a sterile transgenic Teal Tetra.

26. The transgenic tetra of claim 23, wherein the fish is homozygous for at least one of the integrated expression cassettes.

27. The transgenic tetra of claim 23, wherein the fish is heterozygous for at least one of the integrated expression cassettes.
